(19) United States
(12) Patent Application Publication
(10) Pub. No.: US 2024/0139281 A1
(43) Pub. Date: May 2, 2024

(54) USE OF GOJI GLYCOPEPTIDE IN PREPARATION OF DRUG FOR PREVENTING AND/OR TREATING AMYOTROPHIC LATERAL SCLEROSIS

(71) Applicants: Weidong Le, Houston, TX (US); Guohui SU, Hong Kong (CN); NINGXIA QIPEPTIDE TECHNOLOGY CO., LTD, Zhongwei (CN)

(72) Inventors: Weidong LE, Houston, TX (US); Guohui SU, Hong Kong (CN); Xiaolan XU, Chengdu (CN); Xiaojiao XU, Chengdu (CN); Libing ZHOU, Guangzhou (CN); Li ZHANG, Guangzhou (CN); Zhexiong YU, Zhongwei (CN); Jinxia WANG, Zhongwei (CN); Fu FAN, Zhongwei (CN)

(73) Assignees: Weidong LE, Houston, TX (US); Guohui SU, Hong Kong (CN); NINGXIA QIPEPTIDE TECHNOLOGY CO., LTD, Zhongwei (CN)

(21) Appl. No.: 18/236,085

(22) Filed: Aug. 21, 2023

Related U.S. Application Data
(63) Continuation-in-part of application No. PCT/CN2022/112447, filed on Aug. 15, 2022.

(30) Foreign Application Priority Data
Sep. 8, 2021 (CN) 202111047838.2

(51) Int. Cl. A61K 38/14; A61K 36/815; A61P 21/00

(52) U.S. Cl. CPC A61K 38/14; A61K 36/815; A61P 21/00

(57) ABSTRACT

The present invention relates to the field of biomedicine, and in particular to a use of goji glycopeptide in the preparation of a drug for preventing and/or treating amyotrophic lateral sclerosis. Experimental results prove the use of goji glycopeptide in the preparation of a drug for preventing and/or treating amyotrophic lateral sclerosis.

This application is a continuation-in-part application based upon International Patent Application No. PCT/CN2022/112447 filed Aug. 15, 2021, which claims the priority of Chinese Patent Application No. 202111047838.2, filed with the China National Intellectual Property Administration on Sep. 8, 2021, and titled with “USE OF GOJI GLYCOPEPTIDE IN PREPARATION OF DRUG FOR PREVENTING AND/OR TREATING AMYOTROPHIC LATERAL SCLEROSIS”, the disclosures of each of which are hereby incorporated by reference in their entirety.

### FIELD

The present disclosure relates to the field of biomedicine, and in particular to use of Lycium barbarum glycopeptide in the manufacture of a medicament for preventing and/or treating amyotrophic lateral sclerosis.

### BACKGROUND

Amyotrophic lateral sclerosis (ALS) is a complex disease involving multiple molecular mechanisms, and its etiology and pathogenesis have not yet been elucidated. Studies have found that the pathogenesis is related to nucleocytoplasmatic transport defects, abnormal RNA metabolism and RNA-binding proteins, abnormal protein aggregation, impaired DNA repair, mitochondrial dysfunction and oxidative stress, oligodendrocyte dysfunction, microglial dysfunction, defective axonal transport, defective vesicle transport, excitotoxicity, etc.

With the advancement of science and technology, a deeper understanding of the etiology and pathogenesis of amyotrophic lateral sclerosis has been gained. Scientists have developed numerous treatment methods based on pathogenesis, including drug therapy, gene therapy, and stem cell therapy. Among them, only riluzole and edaravone have been approved for clinical use in the treatment of amyotrophic lateral sclerosis. Riluzole, belonging to the class of benzothiazoles, is a glutamate inhibitor that can interfere with NMDA (N-methyl-D-aspartate) receptor-mediated responses, continuously regulate sodium channel currents, and block calcium channel currents, thereby reducing the presynaptic release of glutamate. Riluzole has a certain effect on prolonging the survival period of patients, but has little effect on medulla oblongata and limb functions. Edaravone, as a free radical scavenger, has a certain therapeutic effect on amyotrophic lateral sclerosis, but its clinical application is limited by its intravenous injection rather than oral administration and its high price. As for gene therapy and stem cell therapy, there are still great controversies about their efficacy and safety, and they are still in the research stage and have not yet been applied clinically.

It can be seen that the treatment of amyotrophic lateral sclerosis is a challenge in the medical field and a hot spot in the scientific research. The existing treatment methods have many limitations. Therefore, it is of important practical significance to provide a medicament for treating amyotrophic lateral sclerosis.

Lycium barbarum (Chinese name: Ningxia gouqi), also known as wolfberry or gogi berry, is a shrub of the family Solanaceae native to China, with present-day range across Asia and southeast Europe. It is one of the most widely used Chinese herbal medicines (CHMs) and has been associated with health benefits including anti-oxidative stress, anti-tumor, anti-radiation, anti-fatigue, anti-aging, anti-inflammatory and immunomodulatory properties. Lycium barbarum contains various active ingredients, among which, small molecules include carotene/carotenoid, thiamine, riboflavine, nicotinic acid, ascorbic acid, b-sitosterol, zeaxanthin, physalien, betaine, and β-cryptoxanthin; and macromolecules include dietary fibers, Lycium barbarum polysaccharide (LBP), proteins, and fats.

### SUMMARY

In view of this, the present disclosure provides use of Lycium barbarum glycopeptide in the manufacture of a medicament for preventing and/or treating amyotrophic lateral sclerosis.

In order to achieve the above purpose of the present disclosure, the present disclosure provides the following technical solutions:

The present disclosure provides use of Lycium barbarum glycopeptide in the manufacture of a medicament for preventing and/or treating amyotrophic lateral sclerosis.

The present disclosure provides a method for preventing and/or treating amyotrophic lateral sclerosis, comprising administering to a subject in need thereof Lycium barbarum glycopeptide.

In some specific embodiments of the present disclosure, the Lycium barbarum glycopeptide is at a dosage of 1 mg/kg animal body weight to 100 mg/kg animal body weight.

In some specific embodiments of the present disclosure, the Lycium barbarum glycopeptide is at a dosage of 20 mg/kg animal body weight.

In some specific embodiments of the present disclosure, the Lycium barbarum glycopeptide slows down the weight loss of SOD1G93A transgenic mice.

In some specific embodiments of the present disclosure, the Lycium barbarum glycopeptide prolongs the survival period of SOD1G93A transgenic mice.

In some specific embodiments of the present disclosure, the Lycium barbarum glycopeptide prolongs the disease course of SOD1G93A transgenic mice.

The present disclosure provides a method for down-regulating the relative expression level of an inflammatory factor, comprising administering to a subject in need thereof Lycium barbarum glycopeptide.

In some specific embodiments of the present disclosure, the inflammatory factor is selected from the group consisting of IL-1β, IL-6, TNF-α, and a combination thereof.

The present disclosure provides a method for promoting the activation of microglia to M2 type, inhibiting the activation of microglia to M1 type, or up-regulating the expression of IL-10, comprising administering to a subject in need thereof Lycium barbarum glycopeptide.

The present disclosure also provides a method for preventing and/or treating amyotrophic lateral sclerosis, comprising administering Lycium barbarum glycopeptide. In some specific embodiments of the present disclosure, the Lycium barbarum glycopeptide is administered at a dosage of 1 mg/kg animal body weight to 100 mg/kg animal body weight. In some specific embodiments of the present disclosure, the Lycium barbarum glycopeptide is at a dosage of 20 mg/kg animal body weight.

The present disclosure provides use of Lycium barbarum glycopeptide in the manufacture of a medicament for preventing and/or treating amyotrophic lateral sclerosis. Experimental results demonstrate the use of Lycium barbarum glycopeptide in the manufacture of a medicament for preventing and/or treating amyotrophic lateral sclerosis. Specifically, Lycium barbarum glycopeptide slows down the weight loss of SOD1G93A transgenic mice, prolongs the survival period of SOD1G93A transgenic mice, prolongs the disease course of SOD1G93A transgenic mice, and down-regulates the relative expression level of inflammatory factors in the lumbar spine of SOD1G93A transgenic mice.

### BRIEF DESCRIPTION OF DRAWINGS

In order to more clearly illustrate the embodiments of the present disclosure or the technical solutions in the prior art, the following briefly introduces the drawings that are required in the description of the embodiments or the prior art.

FIG. 1 shows the technical route of the present disclosure.

FIG. 2 shows the body weight curve of the mice in the Tg-LBP and Tg-Con groups.

FIG. 3 shows that Lycium barbarum glycopeptide prolongs the survival period and disease course of SOD1^{G93A} mice. Kaplan-Meier survival analysis curves indicate the changes in survival period (A) (B), onset time (C) (D) and disease course (E) (F) of the mice in the Lycium barbarum glycopeptide group and normal saline group. Log-rank test analysis shows that compared with the mice in the Tg-Con group, the average survival period of the mice in the Tg-LBP group was prolonged by 39 days (199.2±10.3 vs 160.5±12.2, *p<0.05), and moreover, the average disease course of the mice in the Tg-LBP group was prolonged by 22 days (53.3 4.3 vs 31.2±3.0, **p<0.01).

FIG. 4 shows the effects of Lycium barbarum glycopeptide on IL-1β (A), IL-6 (B) and TNF-α (C) in the lumbar spine of SOD1G93A mice.

FIG. 5 shows the technical route of Example 5.

FIG. 6 shows the effects of Lycium barbarum glycopeptide treatment on the onset time, survival period and disease course of SOD1^{G93A} mice; where (a) shows the Kaplan-Meier statistical analysis of the onset time of SOD1^{G93A} mice in the administration group and control group; (b) shows the comparison of onset age of SOD1^{G93A} mice in the administration group and control group; (c) shows the Kaplan-Meier statistical analysis of the survival period of SOD1^{G93A} mice in the administration group and control group; (d) shows the comparison of onset age of SOD1^{G93A} mice in the administration group and control group; (e) shows the Kaplan-Meier statistical analysis of the disease course of SOD1^{G93A} mice in the administration group and the control group; and (f) shows the comparison of the disease course of SOD1^{G93A} mice in the administration group and control group; with data expressed as mean±SEM, n=10 for each group, **p<0.01, ***p<0.001.

FIG. 7 shows the effects of Lycium barbarum glycopeptide on the astrocytes of SOD1^{G93A} mice; where (a) shows the immunofluorescence staining images of GFAP in the spinal cord astrocytes of the mice in the WT-NS, WT-LbGp, TG-NS, and TG-LbGp groups; and (b) shows the quantitative analysis of mean fluorescence density per unit area of GFAP immunofluorescence staining; with data expressed as mean±SEM, n=3 for each group; ***p<0.001 compared with WT-NS; ###p<0.001 compared with WT-LbGP; &&p<0.01 compared with TG-NS.

FIG. 8 shows the effects of Lycium barbarum glycopeptide on the microglia of SOD1^{G93A} mice; where (a) shows the immunofluorescence staining of Iba-1 in spinal cord microglia of mice in the four groups; and (b) shows the quantitative analysis of mean fluorescence density per unit area of Iba-1 immunofluorescence staining; with data expressed as mean±SEM, n=3 for each group; ****p<0.0001 compared with WT-NS, ####p<0.0001 compared with WT-LbGP, &&p<0.01 compared with TG-NS.

FIG. 9 shows the effects of Lycium barbarum glycopeptide on the relative expression level of marker mRNA of M1/M2 microglial in the lumbar spine of SOD1^{G93A} mice; where (a) shows the relative expression level of CD86 mRNA in the spinal cord tissues of the mice in the four groups; (b) shows the relative expression level of iNOS mRNA in the spinal cord tissues of the mice in the four groups; and (c) shows the relative expression level of CD206 mRNA in the spinal cord tissues of the mice in the four groups; with data expressed as mean±SEM, n=3 for each group; **p<0.01, ***p<0.001 compared with WT-NS; #p<0.05, ##p<0.01, ###p<0.001 compared with WT-LbGP; &p<0.05, &&p<0.01 compared with TG-NS;

FIG. 10 shows the effects of Lycium barbarum glycopeptide on the relative expression level of the mRNA of inflammatory factors in the lumbar spine of SOD1^{G93A} mice; where (a) shows the relative expression level of IL-6 mRNA in the spinal cord of the mice in the four groups; (b) shows the relative expression level of TNF-α mRNA in the spinal cord of the mice in the four groups; (c) shows the relative expression level of IL-1β mRNA in the spinal cord of the mice in the four groups; (c) shows the relative expression level of IL-10 mRNA in the spinal cord of the mice in the four groups; with data expressed as mean±SEM, n=3 for each group; ****p<0.0001, ***p<0.001 compared with WT-NS; ##p<0.01, ###p<0.001, ####p<0.0001 compared with WT-LbGP; &p<0.05, &&p<0.01 compared with TG-NS.

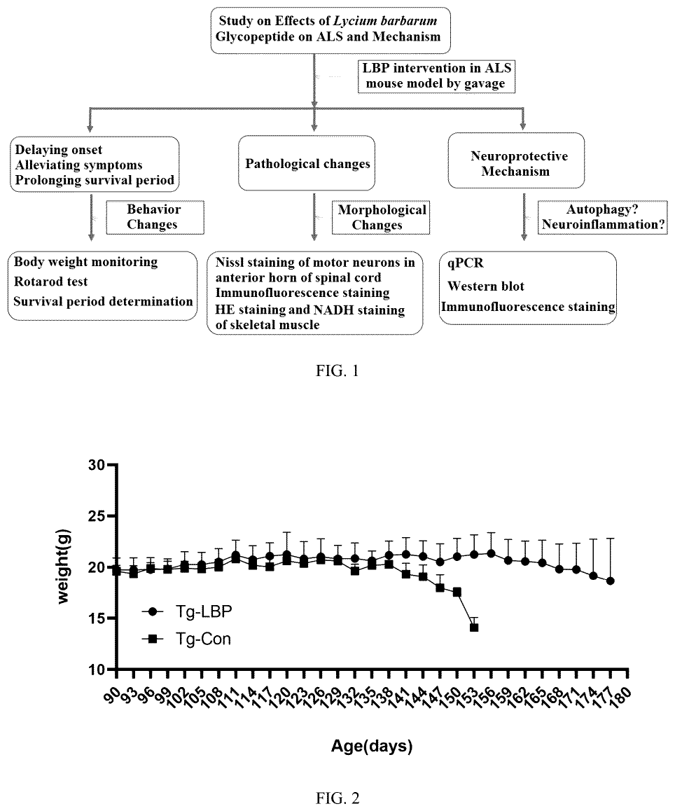

### DETAILED DESCRIPTION

The present disclosure discloses use of Lycium barbarum glycopeptide in the manufacture of a medicament for preventing and/or treating amyotrophic lateral sclerosis. Those skilled in the art can learn from the content of the present disclosure and appropriately modify the process parameters for realization. In particular, it should be noted that all similar replacements and modifications are apparent to those skilled in the art, and are all considered to be included in the present disclosure. The methods and uses of the present disclosure have been described through preferred embodiments, and the skilled in the art can apparently make modifications or appropriate changes and combinations to the methods and uses described herein without departing from the content, spirit and scope of the present disclosure to realize and apply the techniques of the present disclosure.

Explanation of terms: The mixture of several polysaccharide conjugates extracted from Lycium barbarum is called wolfberry glycopeptides, Lycium barbarum glycopeptides, Lycium barbarum glycopeptide composition or Lycium barbarum extraction, which is also referred to as LBP or LbGp in some experiments. LBP is a glycopeptide composition extracted from the fruits of Lycium barbarum, comprising primarily glucose, arabinose, galactose, rhamnose, mannose, and xylose. It is reported that there are at least five glycopeptides determined by column chromatography in the extraction, LbGp1 (LB1), LbGp2 (LB2), LbGp3 (LB3), LbGp4 (LB4) and LbGp5 (LB5).

U.S. Ser. No. 11/110,144B2 discloses a novel and environmentally friendly method for preparing a glycopeptide composition from wolfberry without organic solvent extraction or precipitation, the method comprising: (a) soaking fruit of wolfberry in water and centrifuging to remove precipitated solids to obtain a first extract solution; (b) heating the first extract solution to provide a flocculation in the first extract solution, and centrifuging the first extract solution to remove the flocculation to obtain a second extract solution, wherein the second extract solution has a light transmittance at 50% or higher at 400 nm; and (c) treating the second extract solution with an ultrafiltration membrane, obtaining a cut-off solution with a molecular weight cutoff of the ultrafiltration membrane, concentrating, and drying the cut-off solution to obtain a glycopeptide composition, wherein the flocculation is formed by agglomerating insoluble substances in the first extract solution into precipitates, the molecular weight cutoff of the ultrafiltration membrane is in a range of 1000 Da to 2000 Da, and each of the steps (1) to (3) is conducted in water only. Preferably, the fruit wolfberry is soaked in water at a temperature in a range of 10° C. to 35° C. for 2 hours to 10 hours. Preferably, the first extract solution is heated at a temperature in a range of 45° C. to 70° C. for 0.5 hour to 5 hours to form the flocculation. Preferably, the mass ratio of the fruit of wolfberry to water for soaking in step (a) is 1:1 to 1:15; more preferably, the fruit of wolfberry is dried fruit and the mass ratio of the dried fruit to the amount of the water for soaking is 1:5 to 1:15, or the fruit of wolfberry is fresh fruit and the mass ratio of the fresh fruit to the amount of the water for soaking is 1:1 to 1:3. Preferably, centrifuging to obtain the first extract solution is at a centrifugal speed of 1000 rpm to 4000 rpm for 10 seconds to 1 minute. Preferably, the light transmittance of the second extract solution is at 60% or more at 400 nm. Preferably, the first extract solution is heated to a temperature in a range of 45° C. to 70° C. for a time period of 0.5 hour to 5 hours, and centrifuged at a centrifugal speed of 6000 rpm to 16000 rpm for 5 seconds to 5 minutes. Preferably, the cut-off solution is dried by freeze drying, spray drying, or a combination thereof, to obtain the glycopeptide composition. Preferably, the method further comprises continuously providing water to the cut-off solution during ultrafiltration in step (c); and monitoring electrical conductivity and sugar degree of the cut-off solution; wherein the cut-off solution is collected when the electrical conductivity of the cut-off solution is below 1000 us/cm and the sugar degree is below 1.2.

In the use of Lycium barbarum glycopeptide in the manufacture of a medicament for preventing and/or treating amyotrophic lateral sclerosis of the present disclosure, the raw materials and reagents used are all commercially available. Among them, Lycium barbarum glycopeptide was purchased from Ningxia Tianren Goji Biotechnology Co., Ltd. (Cat No. 0134).

Grouping and Administration of Experimental Animals

Lycium barbarum glycopeptide (from Ningxia Tianren Goji Biotechnology Co., Ltd.) was dissolved in normal saline to a final concentration of 2 mg/ml, and then administered by gavage at 0.01 ml/g body weight at a concentration of 20 mg/Kg d.

Ten SOD1G93A female mice:

Tg-LBP group (n=5), Lycium barbarum glycopeptide administered by gavage at 20 mg/Kg d;

Tg-Con group (n=5), normal saline administered by gavage at 0.01 ml/g d.

The following further illustrates the present disclosure in conjunction with examples:

### Example 1 Preparation of the Glycopeptide Composition with Dried Fruits

The Lycium barbarum glycopeptide composition was prepared according to the method method disclosed in U.S. Ser. No. 11/110,144B2. Specifically, it was performed as follows:

Dried fruits of wolfberry 100 g is smashed and soaked in deionized water. The amount of deionized water for soaking is at a mass ratio of 15 times to the amount of dried fruits, and the soaking is conducted at 10° C. for 10 hours. Then, the soaking liquid is placed in a CR22G centrifuge and centrifuged at 1000 rpm for 1 minute, and the supernatant obtained by centrifugation is observed to be turbid.

The supernatant is placed in a water bath and heated to 40° C. for 5 hours. The pulp and pectin remained in the supernatant congregate to a flocculation. The liquid containing the congregated flocculation is placed in a CR22G centrifuge and centrifuged at 16,000 rpm for 5 seconds, and a clear solution is obtained. The light transmittance of the clear solution at 400 nm is 83% as measured by an N4S UV-visible spectrophotometer.

The clear solution is placed in an 1812 ultrafiltration device for ultrafiltration. The molecular weight cutoff of the ultrafiltration membrane is 1000 Da and the working pressure is 5 kg. Deionized water is continuously supplemented to the cut-off solution during ultrafiltration. The change of conductivity is monitored online by DDSJ-318 conductivity meter and real-time change in sugar degree is monitored by PAL-1 Brix meter. When the conductivity of the cut-off solution is decreased to 500 us/cm and the sugar degree is decreased to 0.7, the solution containing a macromolecular portion cut-off by the ultrafiltration membrane is collected, concentrated, and freeze-dried to obtain 0.85 g of wolfberry glycopeptide composition.

The wolfberry glycopeptide product is analyzed by HPLC. The portion with a molecular weight of 1000-10000 Da accounts for 80% in the product; the protein content is at 35% weight percentage as determined by the Kjeldahl method, neutral polysaccharide content is at 20% weight percentage as determined by the anthrone-sulfuric acid method, and uronic acid content is at 20% weight percentage as determined by carbazole method.

### Example 2 Effects of Lycium barbarum Glycopeptide on Body Weight of SOD1^{G93A} Transgenic Mice

Mutations in copper-zinc superoxide dismutase 1 (SOD1) are a known genetic cause of ALS. SOD1^{G93A} transgenic mice express a G93A mutant form of human SOD1, and have been used extensively to investigate molecular mechanisms in ALS. In the present disclosure, SOD1^{G93A} transgenic mice were used to demonstrate the effects of Lycium barbarum glycopeptide, and can be purchased from, for example, The Jackson Laboratory.

The mice were weighed every 3 days from the age of 90 days to observe the effects of Lycium barbarum glycopeptide on the body weight of SOD1^{G93A} mice. From the body weight curve of the mice in FIG. 2, it can be observed that the body weight of the mice in the Tg-Con group and the Tg-LBP group decreased significantly after the onset of the disease. Compared with the mice in the Tg-Con group, the time for the body weight loss of the mice in the Tg-LBP group was later, and the rate of the body weight loss was slower. The results suggest that Lycium barbarum glycopeptide had a tendency to slow down the weight loss of SOD1^{G93A} transgenic mice with a significant statistical difference (p<0.01).

TABLE 1
Effects of Lycium barbarum glycopeptide on body weight of
SOD1^{G93A} transgenic mice

| | Tg-LBP | Tg-Con |
|---|---|---|
| 90 | 19.74 ± 0.95 | 19.60 ± 0.29 |
| 93 | 19.74 ± 0.97 | 19.35 ± 0.4 |
| 96 | 19.78 ± 0.96 | 19.88 ± 0.29 |
| 99 | 19.82 ± 0.89 | 19.80 ± 0.39 |
| 102 | 20.26 ± 1.01 | 19.90 ± 0.34 |
| 105 | 20.26 ± 0.97 | 19.83 ± 0.30 |
| 108 | 20.5 ± 1.03 | 20.00 ± 0.37 |
| 111 | 21.18 ± 1.09 | 20.8 ± 0.21 |
| 114 | 20.72 ± 1.05 | 20.18 ± 0.38 |
| 117 | 21.08 ± 1.01 | 20.05 ± 0.42 |
| 120 | 21.02 ± 1.27 | 20.6 ± 0.38 |
| 123 | 20.8 ± 1.2 | 20.35 ± 0.21 |
| 126 | 21 ± 1.22 | 20.7 ± 0.07 |
| 129 | 20.8 ± 1.02 | 20.6 ± 0.2 |
| 132 | 20.84 ± 1.13 | 19.63 ± 0.33 |
| 135 | 20.62 ± 0.87 | 20.18 ± 0.31 |
| 138 | 21.16 ± 1.05 | 20.28 ± 0.43 |
| 141 | 21.24 ± 1.17 | 19.33 ± 0.53 |
| 144 | 21.04 ± 1.11 | 19.08 ± 0.58 |
| 147 | 20.5 ± 1.22 | 17.98 ± 0.65 |
| 150 | 21.02 ± 1.24 | 17.5 ± 0.23* |
| 153 | 21.22 ± 1.3 | 14.08 ± 0.5** |

All data are expressed as mean ± SEM, and the statistical analysis was performed using GraphPad 8.0, *p < 0.05, **p < 0.01.

### Example 3 Effects of Lycium barbarum Glycopeptide on Onset Time and Survival Period of SOD1G93A Transgenic Mice

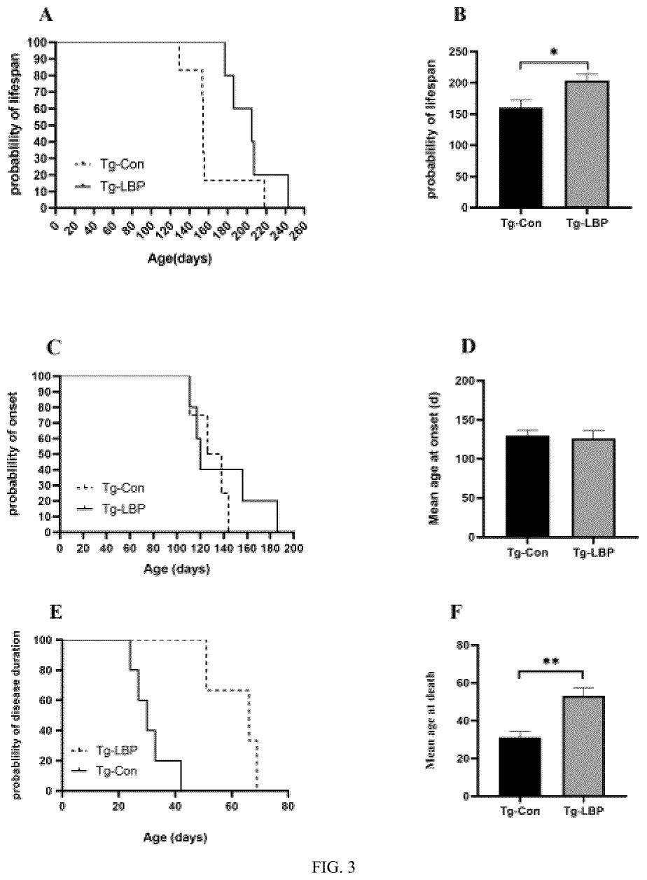

The mice were subjected to rotarod test every 3 days from the age of 90 days, and the onset time of the mice was evaluated according to the rotarod performance and body weight changes of the mice. The time of death of the mice was determined according to the righting reflex of the mice in the terminal stage of the disease, so as to evaluate the survival period of the mice. The experimental results in FIG. 3 suggest that Lycium barbarum glycopeptide prolonged the survival period (n=5, *p<0.05) and disease course (n=5, **p<0.01) of SOD1G93A transgenic mice.

TABLE 2

| | Survival period | Onset time | Disease course |
|---|---|---|---|
| Group | (days) | (days) | (days) |
| Tg-Con group | 160.5 ± 12.2 | 129.8 ± 7.3 | 31.2 ± 3.0 |
| Tg-LBP group | 199.2 ± 10.3* | 132.6 ± 6.9 | 53.3 ± 4.3** |

All data are expressed as mean ± SEM, and the statistical analysis was performed using t-tests (GraphPad 8.0 ), *p < 0.05, **p < 0.01.

### Example 4 Effects of Lycium barbarum Glycopeptide on Inflammatory Factors in Lumbar Spine of SOD1^{G93A} Transgenic Mice

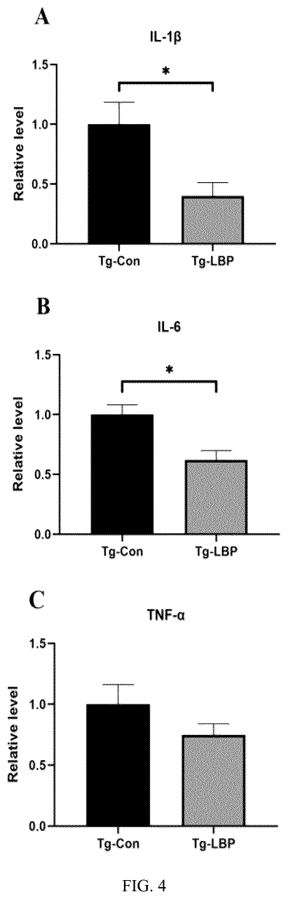

Studies have shown that microglia play an important role in the occurrence and development of ALS. M1 microglia promote neuroinflammation and aggravate neuronal damage by secreting pro-inflammatory factors such as IL-1β, IL-6 and TNF-α. The relative expression level of the mRNA of pro-inflammatory factors (IL-1β, IL-6, TNF-α) in the lumbar spine was detected by RT-PCR. The experimental results shown in FIG. 4 suggest that compared with the mice in the Tg-Con group, the relative expression level of IL-1β mRNA and IL-6 mRNA in the lumbar spine of the mice in the Tg-LBP group decreased significantly with a statistically significant difference, and the relative expression level of TNF-α mRNA had a downward trend with no statistically significant difference. It can be seen that Lycium barbarum glycopeptide can inhibit the expression of pro-inflammatory factors in the lumbar spine of SOD1^{G93A} transgenic mice, and exert a neuroprotective effect by inhibiting neuroinflammation.

TABLE 3

| Group | IL-1β | IL-6 | TNF-α |
|---|---|---|---|
| Tg-Con | 1.0 ± 0.18 | 1.0 ± 0.08 | 1.0 ± 0.16 |
| Tg-LBP | 0.4 ± 0.11* | 0.62 ± 0.07* | 0.74 ± 0.09 |

All data are expressed as mean ± SEM, and the statistical analysis was performed using t-tests (GraphPad 8.0 ), *p < 0.05, **p < 0.01.

### Example 5

1. Grouping and administration of experimental animals:

Lycium barbarum glycopeptide (from Ningxia Tianren Goji Biotechnology Co., Ltd.) was dissolved in normal saline to a final concentration of 2 mg/ml, and then administered by gavage at 0.01 ml/g body weight at a concentration of 20 mg/Kg d.

32 female transgenic (TG) mice were reared in cages, 4 in each cage. 32 transgenic female mice were randomly divided into a Lycium barbarum glycopeptide treatment group (TG-LbGp) and a normal saline control group (TG-NS), 16 in each group. Additionally, 32 wild-type (WT) female mice from the same litter with matched age and sex were randomly divided into a Lycium barbarum glycopeptide treatment group (WT-LbGp) and a normal saline control group (WT-NS), 16 in each group. The specific scheme is as follows: on the 90th day, the mice in the TG-LbGp group and WT-LbGp group were administered with Lycium barbarum glycopeptide solution by gavage at a dosage of 20 mg/kg until the transgenic mice died; and the mice in the TG-NS group and WT-NS group were given normal saline with an equal volume by gavage until the transgenic mice died. At the mice age of 170 days, 6 mice in each group were randomly sacrificed to collect materials for detection of pathological and biochemical indicators, and the remaining 10 mice in each group were used to observe the onset time and survival period.

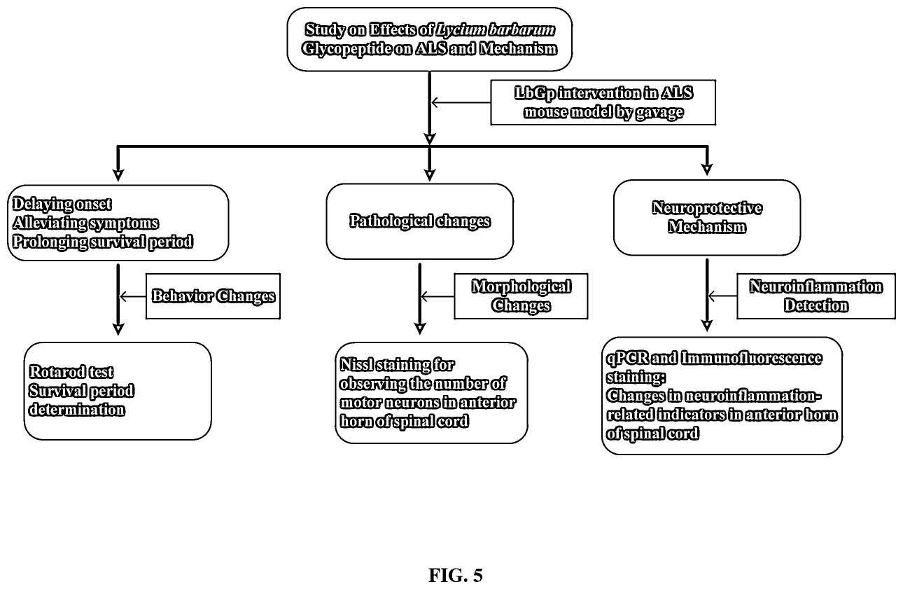

2. Technical route: as shown in FIG. 5.

3. Experimental results:

3.1 Effects of Lycium barbarum Glycopeptide on Onset Time, Survival Period and Disease Course

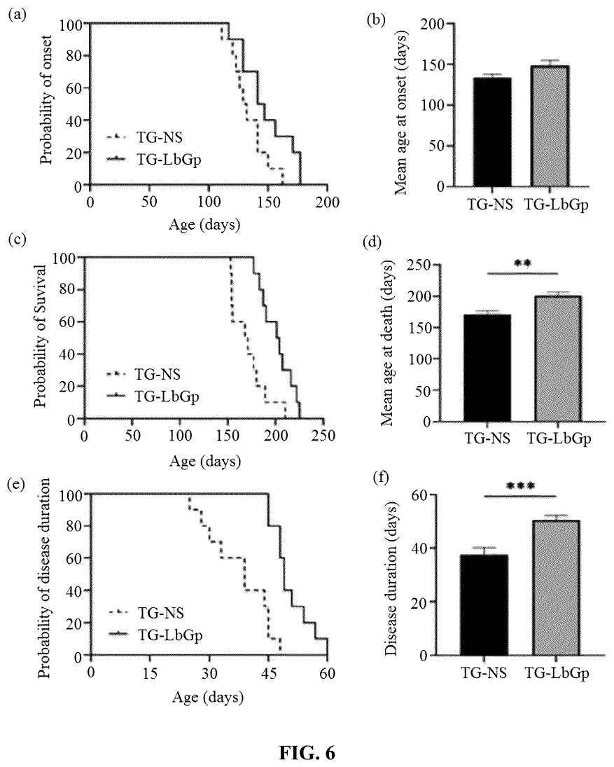

In this study, the mice were subjected to rotarod test to evaluate the onset time. As shown in FIG. 6, after administration of Lycium barbarum glycopeptide, the onset time of the mice in the TG-LbGp group was delayed compared with the mice in the TG-NS group (see FIG. 6a), but there was no statistical difference (133.5±4.801 vs 148.5±6.712, P=0.0858) (FIG. 6b). At the terminal stage of the disease, the time of death of the mice was determined according to the righting reflex of the mice, so as to count the survival period. The research results are shown in FIG. 6 that compared with the mice in the TG-NS group, the average survival period of the mice in the TG-LbGp group was prolonged by about 30 days (171.1±5.896 vs 201.1±5.305, P<0.01) (see FIG. 6c and FIG. 6d). The results of this study suggest that treatment with Lycium barbarum glycopeptide can prolong the lifespan of SOD1^{G93A} transgenic mice by about 17.5%. Disease course refers to the period from onset to death of the transgenic mice, which reflects the progression rate of the disease to a certain extent. By comparing the disease course of the mice in the TG-NS group and the TG-LbGp group, it is found that after administration of Lycium barbarum glycopeptide, the disease course of the mice in the TG-LbGp group was prolonged by about 35.1% compared with the mice in the TG-NS group (37.60±2.566 vs 50.60±1.572, P<0.001) (see FIG. 6e and FIG. 6f). The results suggest that treatment with Lycium barbarum glycopeptide can delay the disease progression. The disease course of the mice in the TG-NS group was prolonged by about 35.1% (37.60±2.566 vs 50.60±1.572, P<0.001) (see FIG. 6e and FIG. 6f). The results suggest that treatment with Lycium barbarum glycopeptide can delay the disease progression.

3.2 Effects of Lycium barbarum Glycopeptide on Astrocytes

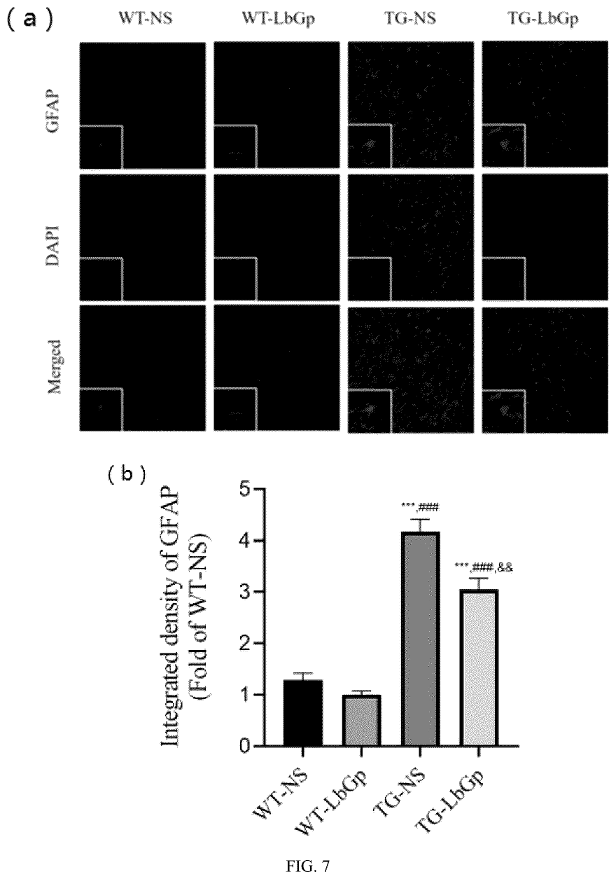

Astrocytes serve multiple functions and are the most common cells in the central nervous system, as well as a key factor for maintaining and supporting the survival of central nervous system motor neurons. Enlarged cell size, increased synapses, and upregulated GFAP expression of astrocytes were found in the spinal cord and motor regions of the brain in both SOD1 mice and human patients with ALS. Activated astrocytes have been found to play a key role in the pathology of ALS through multiple mechanisms in animal models of ALS and patients with ALS. Therefore, regulating the activation of astrocytes may be a potential therapeutic target for ALS. To observe the effects of Lycium barbarum glycopeptide on astrocytes, an anti-GFAP antibody was used to perform immunofluorescence staining on frozen sections of the lumbar spine of SOD1^{G93A} transgenic mice, so as to evaluate the number of astrocytes. The results are shown in FIGS. 3-4 that the GFAP staining-positive astrocytes in the lumbar spine tissue of SOD1^{G93A} transgenic mice were significantly more than those of the wild-type mice. Compared with the mice in the TG-NS group, the GFAP staining-positive astrocytes in the TG-LbGp group were significantly less (see FIG. 7a), and the average fluorescence density per unit area of GFAP in the TG-LbGp group was reduced by about 27.1% (see FIG. 7b), which further proved this result, suggesting that treatment with Lycium barbarum glycopeptide can effectively inhibit astrocyte proliferation in ALS transgenic model mice.

3.3 Effects of Lycium barbarum Glycopeptide on Microglia

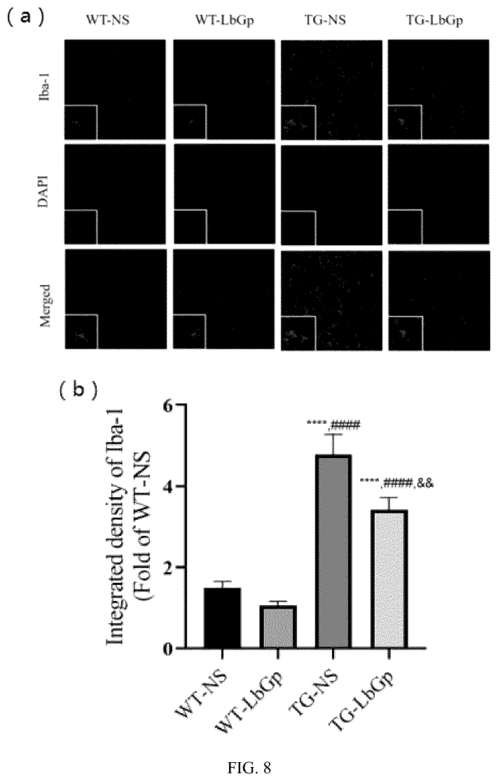

In order to evaluate the effects of Lycium barbarum glycopeptide treatment on microglia, an anti-Iba-1 antibody was used to perform immunofluorescence staining to observe frozen sections of the spinal cord tissue of SOD1^{G93A} mice in this study. The research results are shown in FIG. 8 that the degree of activation of microglia in the lumbar spine tissue of SOD1^{G93A} transgenic mice was significantly higher than that of the wild-type mice. After administration of Lycium barbarum glycopeptide, the Iba-1 staining-positive cells in the lumbar spine of the mice in the TG-LbGp group decreased compared with the mice in the TG-NS group (see FIG. 8a), and the average fluorescence density per unit area of Iba-1 in the TG-LbGp group decreased by 28.5% based on quantitative analysis (see FIG. 8b), suggesting that the degree of activation of microglia significantly decreased. The results suggest that treatment with Lycium barbarum glycopeptide can effectively inhibit the activation of microglia in the ALS transgenic mice.

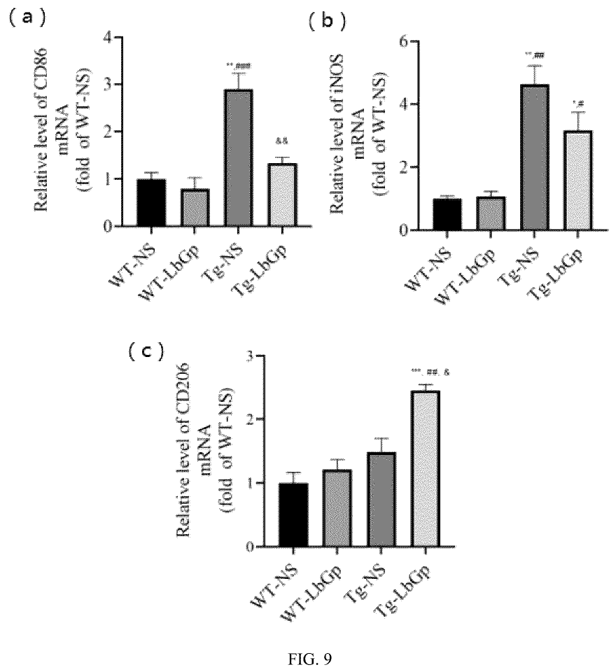

3.4 In this experiment, the relative expression level of the mRNA of M1 microglia markers CD86 and iNOS and M2 microglia marker CD206 in the lumbar spine tissue of the SODIG93A transgenic mouse model was detected by qPCR. The experimental results show (see FIG. 9) that compared with the wild-type mice, the relative expression level of CD86 mRNA in the lumbar spine tissue of the SOD1^{G93A} transgenic mouse model was significantly increased. After administration of Lycium barbarum glycopeptide, the relative expression level of CD86 mRNA of the mice in the TG-LbGp group decreased by 54% compared with the mice in the TG-NS group. After administration of Lycium barbarum glycopeptide, the relative expression level of iNOS mRNA of the mice in the TG-LbGp group also had a downward trend compared with the mice in the Tg-NS group. In addition, compared with the mice in the TG-NS group, the relative expression level of CD206 mRNA in the lumbar spine tissue of the mice in the TG-LbGp group increased by about 66.1%. The above experimental results suggest that Lycium barbarum glycopeptide may be able to regulate the activation state of microglia to inhibit the activation of microglia to M1 type and promote the activation of microglia to M2 type, thereby increasing the proportion of M2 microglia and alleviating the disease progression of ALS.

3.5 Effects of Lycium barbarum Glycopeptide on Inflammatory Factors in Spinal Cord

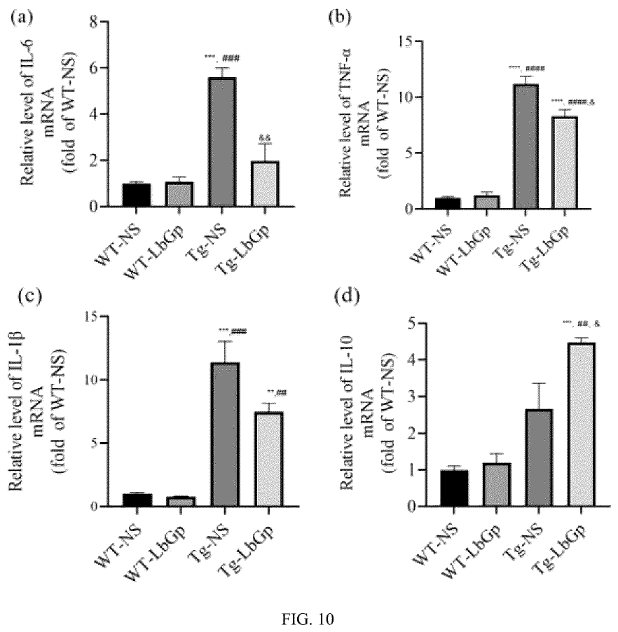

The current study shows that in cases of neuronal injury or other damage, microglia will be activated to secrete pro-inflammatory factors that enhance cytotoxicity (such as IL-1β, IL-6 and TNF-α) or anti-inflammatory neuroprotective factors (such as IL-10) depending on the type and intensity of the stimulus. The neuroinflammatory responses mediated by the activated microglia in the SOD1^{G93A} mouse model play an important role in the occurrence and progression of ALS. In this study, the relative expression level of the mRNA of inflammatory factors in the segments L4-5 of the spinal cord of the SOD1^{G93A} mouse model was detected by qPCR. The experimental results are shown in FIG. 10 that compared with the wild-type mice, the relative expression level of the mRNA of pro-inflammatory factors IL-6, IL-1β and TNF-α in the segments L4-5 of the spinal cord tissue of SOD1^{G93A} transgenic mice was significantly increased. After administration of Lycium barbarum glycopeptide, the relative expression level of the mRNA of IL-6 and TNF-α of the mice in the TG-LbGp group decreased by 64.8% and 25.9%, respectively, compared with the mice in the TG-NS group, and the relative expression level of IL-1β mRNA also had a downward trend, but the difference was not statistically significant. After administration of Lycium barbarum glycopeptide, the relative expression level of IL-10 mRNA of the mice in the TG-LbGp group increased by 68.2% compared with the mice in the TG-NS group. The above results suggest that treatment with Lycium barbarum glycopeptide may inhibit the production of pro-inflammatory factors, thereby exerting a therapeutic effect.

The use of Lycium barbarum glycopeptide in the manufacture of a medicament for preventing and/or treating amyotrophic lateral sclerosis provided by the present disclosure has been introduced in detail above. Specific examples are used herein to illustrate the principle and implementation of the present disclosure. The description of the above examples is only used to help understand the method and core idea of the present disclosure. It should be noted that for those skilled in the art, without departing from the principles of the present disclosure, several improvements and modifications can be made to the present disclosure, and these improvements and modifications also fall within the protection scope of the claims of the present disclosure.

1. A method for treating amyotrophic lateral sclerosis, comprising administering to a subject in need thereof Lycium barbarum glycopeptide composition.

2. The method according to claim 1, wherein the Lycium barbarum glycopeptide is administered at a dosage of 1 mg/kg body weight to 100 mg/kg body weight.

3. The method according to claim 2, wherein the Lycium barbarum glycopeptide is administered at a dosage of 20 mg/kg body weight.

4. A method for down-regulating the relative expression level of an inflammatory factor, comprising administering to a subject in need thereof Lycium barbarum glycopeptide.

5. The method according to claim 4, wherein the inflammatory factor is selected from the group consisting of IL-1β, IL-6, TNF-α, and a combination thereof.

6. A method for promoting the activation of microglia to M2 type, inhibiting the activation of microglia to M1 type, or up-regulating the expression of IL-10, comprising administering to a subject in need thereof Lycium barbarum glycopeptide.
